(19) United States
(12) Patent Application Publication
(10) Pub. No.: US 2020/0306103 A1
(43) Pub. Date: Oct. 1, 2020

(54) INCONTINENCE ARTICLE

(71) Applicant: Audree Blaikie, Advance, NC (US)

(72) Inventor: Audree Blaikie, Advance, NC (US)

(21) Appl. No.: 16/368,012

(22) Filed: Mar. 28, 2019

(51) Int. Cl. A61F 13/471; A61F 13/514; A61F 13/56

(52) U.S. Cl. CPC A61F 13/471; A61F 2013/15146; A61F 13/5611; A61F 13/51476

(57) ABSTRACT

An incontinence article configured for use with an undergarment for absorbing urine leakage caused by stress incontinence. The article includes a base layer and a plurality of pleats extending from a top side of the base layer on each side of the base layer. The article is formed of a flexible and absorbent fabric. The bottom end of the base layer is sealed to the bottom end of the pleats on either side of the base layer. Adhesive on the bottom of the base layer configures the article for adherence to the inside of an undergarment. When base layer is secured to an inside panel of the undergarment, the article expands, via the plurality of pleats, to the contours of the undergarment body of a user wearing the undergarment.

The present invention is directed towards an incontinence article for use in connection with male stress incontinence.

Male stress incontinence is a common medical issue wherein the male's bladder and urethra are not able to properly hold urine, particularly when under pressure such as when the male coughs or sneezes. While there is an abundant market for female stress incontinence products, many of which are similar to female hygiene products, there are limited options for male incontinence products, most of which are bulky and uncomfortable for the user.

Thus, there is a need in the art for an incontinence article for use with male stress incontinence that is effective, yet discrete and comfortable.

### BRIEF SUMMARY

In view of the foregoing background, example implementations of the incontinence article of the present disclosure include a base layer and a plurality of pleats extending from a top side of the base layer on each side of the base layer. The article is formed of a flexible and absorbent fabric. The base layer and pleats may be formed of a unitary fabric. The bottom end of the base layer is sealed to the bottom end of the pleats on each side of the base layer. Adhesive on the bottom of the base layer configures the article for adherence to the inside of an undergarment. When the base layer is secured to an inside panel of the undergarment, the article expands, via the plurality of pleats, to the contours of the undergarment or body of a user wearing the undergarment.

### BRIEF DESCRIPTION OF THE FIGURES

FIG. 1 is a perspective top view of an incontinence article in a flattened state according to a first implementation of the present disclosure.

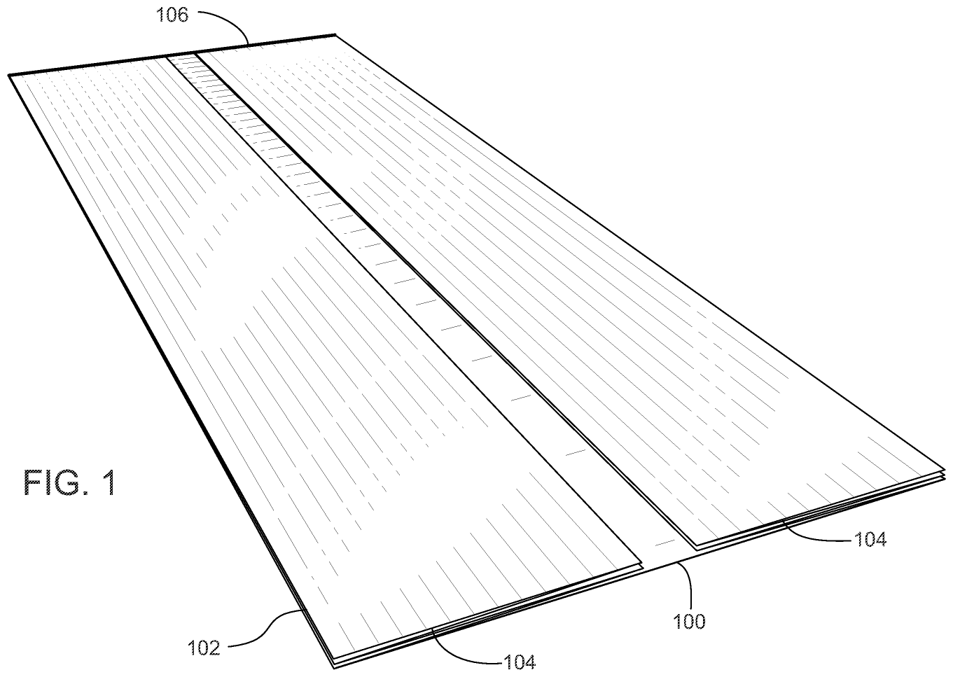

FIG. 2 is a top view of the article of FIG. 1 in a flattened state.

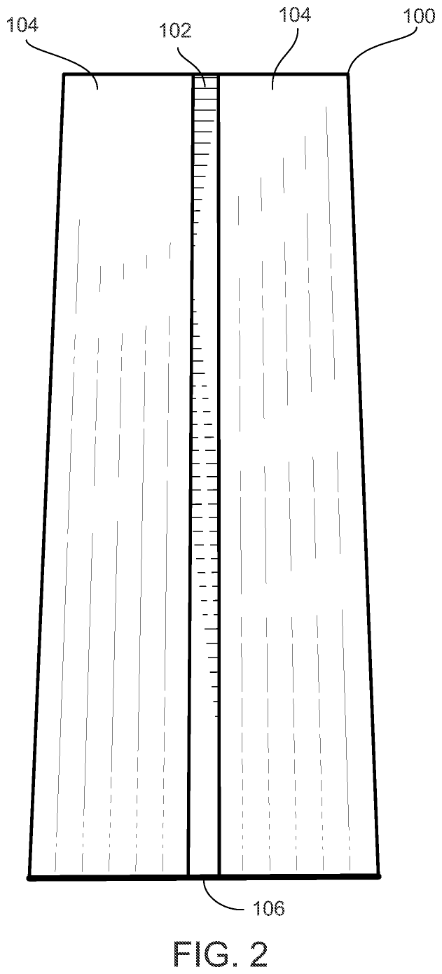

FIG. 3 is a side view of the article of FIG. 1 in a flattened state.

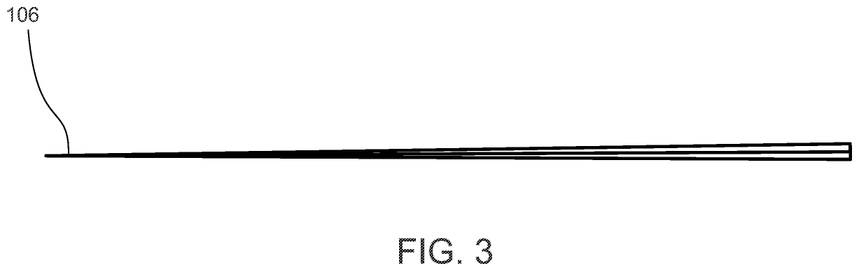

FIG. 4 is a bottom view of the article of FIG. 1.

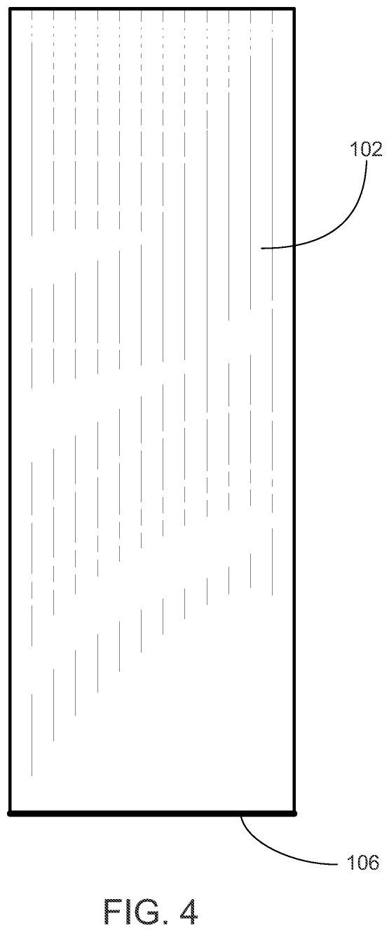

FIG. 5 is a perspective top view of the article of FIG. 1 in an expanded state.

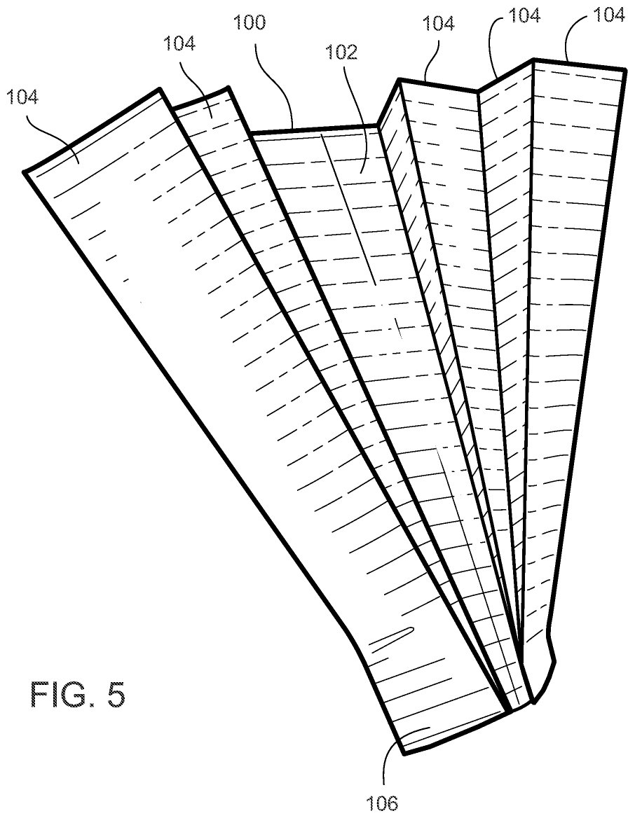

FIG. 6 is a perspective view of the article of FIG. 1 in an expanded state as secured to an inside panel of an undergarment.

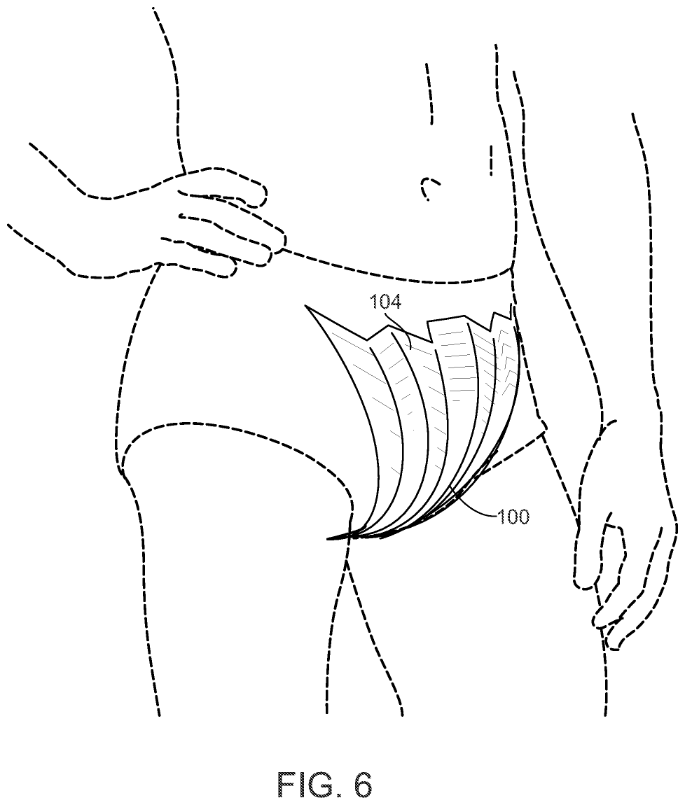

### DETAILED DESCRIPTION OF THE PRESENT INVENTION

The present invention is a discrete, yet effective, absorptive article 100 for use by males who experience stress incontinence. The article 100 is configured for use in connection with the user's undergarment and, as shown in the Figures, the unique pleated construction of the article 100 enables it to easily expand to accommodate the shape and contours of the undergarment while providing significant absorption capacity.

The absorptive article 100 is formed of a flexible and absorbent fabric, such as a laminate with high loft nonwoven fibers infused with super absorbent polymers, which is laminated to a polypropylene backing. The fabric may be treated with a non-toxic odor reducing agent. The unique design includes a base layer 102 with multiple pleats 104 on each side of the base layer 102. The pleated folds 104 on each side may meet (or be close to meeting) in the middle of the base layer 102. In the embodiment shown in the Figures, there are two pleats 104 on each side of the top of the base layer 102. It is noted, however, that the present invention is not limited to a particular number of pleats 104 on either side of the article 100. For example, an article 100 designed for use with a larger person may include more pleats 104 to provide for increased expansion. Additionally, the article 100 is not limited to any particular dimensions; however, sizes may range from about 8″-10″ long and 2½″-3″ a flattened state.

In this implementation of the invention, the base layer 102 and folded pleats 104 on each side are formed of a unitary piece of the fabric such that the pleats 104 are formed by folds and not by seams to litigate the risk of leakage. Also in this implementation of the invention, the folded pleats 104 are sealed to one another and to the base layer 102 at the bottom end 106 of the article 100, such as with a heat seal or glue. Such bottom seal 106 enables the article to expand from the base layer 102 to form a cross-sectional cup shape. An adhesive strip 108 may be applied to the bottom of the base layer including a peel-off cover.

During use, the user peels off the adhesive strip 108 and attaches the article 100 to the inside panel of the front of the user undergarment 120 such that the sealed bottom end 106 is secured towards the bottom of the inside of the front panel of the undergarment 120. The article 100 expands via the pleats 104 within the undergarment 120 to cup the shape of the user's penis, thereby providing a soft, flexible, expandable, and protective shield within the undergarment 120 to absorb urine leakage caused by stress incontinence. Preferably, the absorbent article 100 can absorb up to about 12 cc of urine while keeping the user dry and comfortable. Importantly, the article 100 is inconspicuous and designed to be disposable such that the user can easily and quickly change the article 100 as needed.

While certain embodiments of the invention have been described using specific terms, such description is for present illustrative purposes only, and it is to be understood that changes and variations to such embodiments, including but not limited to the substitution of equivalent features or parts, and the reversal of various features thereof, may be practiced by those of ordinary skill in the art without departing from the spirit or scope of the present disclosure.

What is claimed is:

1. An absorbent article comprising:
a base layer; and
a plurality of pleats extending from a top side of the base layer on each side of the base layer;
wherein a bottom end of the base layer is sealed to a bottom end of at least some of the plurality of pleats on either side of the base layer; and
wherein when the base layer is affixed to an inside panel of an undergarment, the article expands, via the plurality of pleats, to the contours of the undergarment or body of a user wearing the undergarment.

2. The absorbent article of claim 1 wherein the article is formed of a flexible and absorbent fabric.

3. The absorbent article of claim 1 wherein the base layer and plurality of pleats are formed from a unitary fabric.

4. The absorbent article of claim 1 wherein the article further comprises an adhesive strip on a bottom side of the base layer that enables the article to be removably secured to an undergarment.
